(19) United States
(12) Patent Application Publication
(10) Pub. No.: US 2016/0175227 A1
(43) Pub. Date: Jun. 23, 2016

(54) LIQUID ADHESIVE FOR NAIL POLISH

(71) Applicant: MINI MANI MOO, LLC, Frisco, TX (US)

(72) Inventors: Andreina Samaia Landis, Frisco, TX (US); Mark Landis, Frisco, TX (US)

(73) Assignee: MINI MANI MOO, LLC, Frisco, TX (US)

(21) Appl. No.: 14/874,284

(22) Filed: Oct. 2, 2015

Related U.S. Application Data
(63) Continuation-in-part of application No. 14/687,736, filed on Apr. 15, 2015, now Pat. No. 9,149,421.
(60) Provisional application No. 62/094,499, filed on Dec. 19, 2014.

(51) Int. Cl. A61K 8/72; A61K 8/19; A61Q 3/00; A61K 8/27; A61K 8/81; A45D 29/00; A61K 8/92

(52) U.S. Cl. CPC A61K 8/72; A45D 29/004; A61K 8/19; A61K 8/922; A61K 8/27; A61K 8/8141; A61Q 3/00

(57) ABSTRACT

A liquid adhesive for nail polish is a topical composition that is used to facilitate the application and removal of nail polish as well as the creation of intricate nail polish designs. The composition includes rubber latex and water. The liquid adhesive may also include an antiseptic composition of ammonium hydroxide and thiram, a preserving composition of zinc oxide, an aromatic agent, a coloring agent, and a moisturizing agent. The rubber latex forms a liquid-impermeable coating on the surface of the user's skin or nail once applied. This allows a dried coating of the liquid adhesive to be peeled from the skin or nail along with any nail polish applied over the coating. The composition may additionally exhibit antimicrobial and antifungal properties as well as a resistance to decomposition and other unwanted chemical and physical changes.

### CROSS REFERENCE TO RELATED APPLICATIONS AND CLAIM OF PRIORITY

The present application claims priority to U.S. patent application Ser. No. 14/687,736, filed Apr. 15, 2015 entitled “Liquid Adhesive for Nail Polish,” which will issue as U.S. Pat. No. 9,149,421 on Oct. 6, 2015. U.S. patent application Ser. No. 14/687,736 claims priority to U.S. Provisional Patent Application No. 62/094,499 filed on Dec. 19, 2014. The content of both these patent documents is incorporated by reference herein.

### TECHNICAL FIELD

The present disclosure relates generally to a cosmetic product primarily for use with nail polish. More specifically, the present disclosure relates to a liquid adhesive for nail polish that allows the user to reduce mess when applying nail polish, create intricate designs with nail polish, and easily remove nail polish as well.

### BACKGROUND

The cost of a professional manicure or pedicure can often be exorbitant. As such, the best alternative for those who do not wish to spend the time and money required for a professional manicure or pedicure is to apply the nail polish themselves. Because most people do not possess the skills or dexterity required to create intricate nail polish designs, these attempts often result in messy designs with nail polish mistakenly applied to the cuticle rather than solely on the nail. Additionally, the lack of skills and dexterity required generally results in mediocre nail polish designs when compared to professional manicures or pedicures.

### SUMMARY

Embodiments of the present disclosure provide a liquid adhesive for nail polish that facilitates the process of applying nail polish, preparation prior to applying nail polish, and the removal of nail polish. The liquid adhesive may be applied to the cuticle around a nail in order to form a barrier and prevent nail polish from accidentally being applied to the skin. The liquid adhesive may be easily removed by peeling the liquid adhesive off of the skin. Additionally, the liquid adhesive may be applied on top of a nail, allowing the use of multiple layers of nail polish in the creation of intricate nail polish designs.

In one embodiment, a liquid adhesive for nail polish is provided. The liquid adhesive includes a quantity of rubber latex that is between 45% wt. and 70% wt. of the liquid adhesive for nail polish and a quantity of water that is between 20% wt. and 45% wt. of the liquid adhesive for nail polish.

In another embodiment, a liquid adhesive for nail polish is provided. The liquid adhesive includes a quantity of rubber latex that is between 45% wt. and 70% wt. of the liquid adhesive for nail polish and a quantity of water that is between 20% wt. and 45% wt. of the liquid adhesive for nail polish. The quantity of rubber latex and the quantity of water present in the liquid adhesive for nail polish is selected based on environmental conditions.

In yet another embodiment a liquid adhesive for nail polish is provided. The liquid adhesive includes a quantity of rubber latex that is greater than 50% wt. of the liquid adhesive for nail polish and a quantity of water that is less than 50% wt. of the liquid adhesive for nail polish.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 is a block diagram depicting an ingredient composition in a liquid adhesive for nail polish according to an illustrative embodiment of the present disclosure;

FIG. 2 is a block diagram depicting an ingredient composition of an antiseptic composition in a liquid adhesive for nail polish according to an illustrative embodiment of the present disclosure;

FIG. 3 is a block diagram depicting an ingredient composition of a preserving composition in a liquid adhesive for nail polish according to an illustrative embodiment of the present disclosure;

FIG. 4 is a block diagram depicting an ingredient composition of an aromatic agent in a liquid adhesive for nail polish according to an illustrative embodiment of the present disclosure;

FIG. 5 is a block diagram depicting an ingredient composition of a coloring agent in a liquid adhesive for nail polish according to an illustrative embodiment of the present disclosure;

FIG. 6 is a block diagram depicting an ingredient composition of a moisturizing agent in a liquid adhesive for nail polish according to an illustrative embodiment of the present disclosure; and

FIG. 7 is a block diagram depicting weight ratios of an example ingredient composition in a liquid adhesive for nail polish according to an illustrative embodiment of the present disclosure.

### DETAILED DESCRIPTION

The various figures and embodiments used to describe the principles of the present disclosure in this patent document are by way of illustration only and should not be construed in any way to limit the scope of the present disclosure. Those skilled in the art will understand that the principles of the present disclosure may be implemented in any type of suitably-arranged device or system.

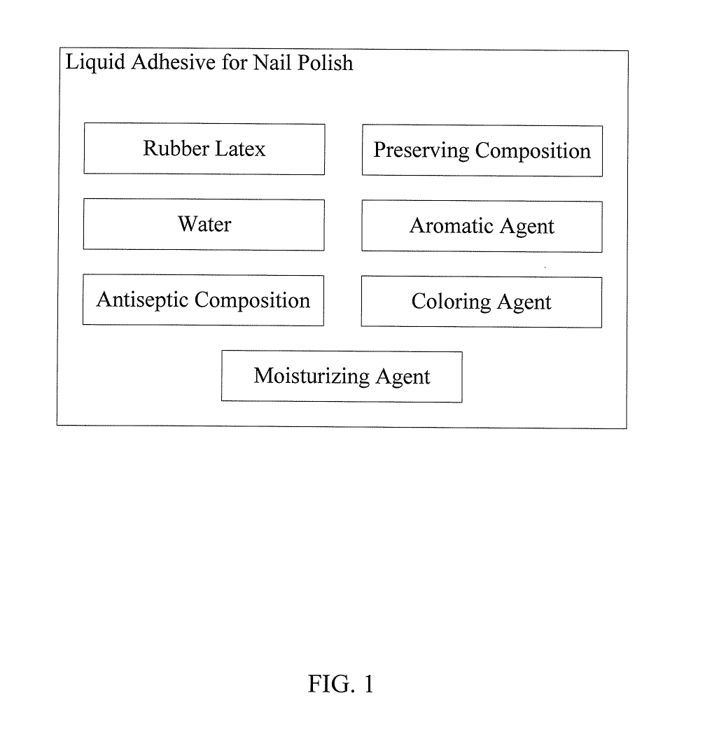

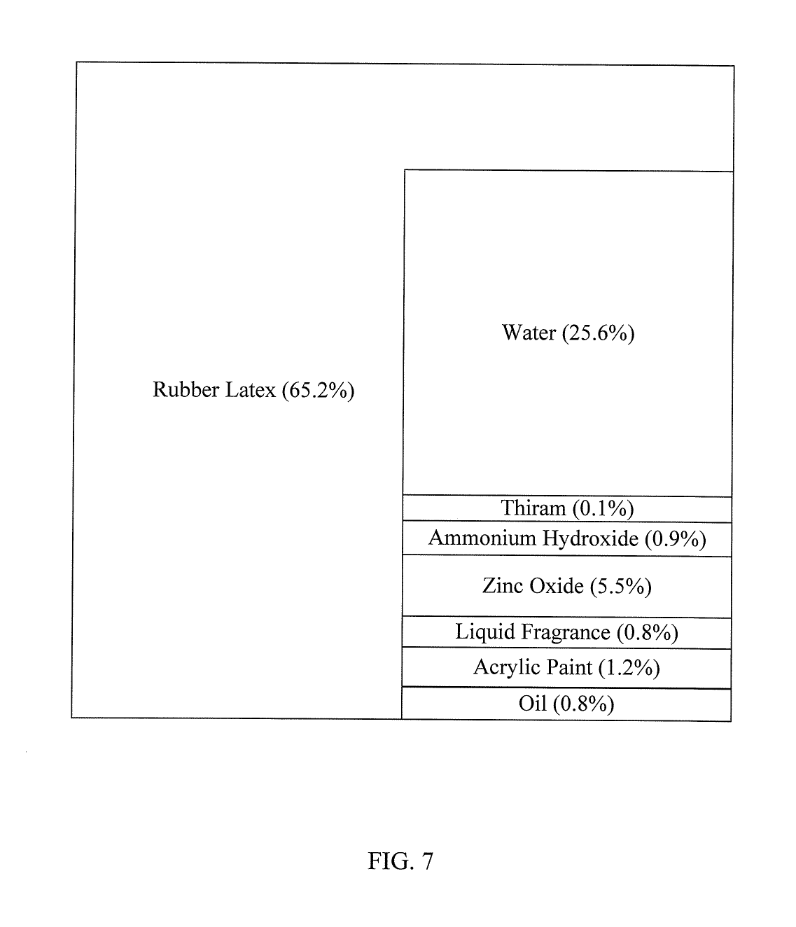

Various embodiments of present disclosure provide a liquid adhesive for nail polish that allows the user to more easily apply nail polish as well as facilitates the creation of intricate nail polish designs and the process of removing nail polish. With reference to FIGS. 1-6, in various embodiments of present disclosure, the liquid adhesive for nail polish comprises a quantity of rubber latex, a quantity of water, an antiseptic composition, a preserving composition, an aromatic agent, a coloring agent, and a moisturizing agent. The specific weight ratios of the liquid adhesive for nail polish in one illustrative embodiment of the present disclosure are shown in FIG. 7.

Again with reference to FIG. 1 and FIG. 7, the quantity of rubber latex serves to provide a liquid-impermeable coating on the surface of the user's skin as well as the surface of the user's nail once applied. Once the liquid adhesive has been applied to the user's skin or nail in liquid form, the liquid adhesive is able to dry. Upon drying, the quantity of rubber latex forms a coating upon the user's skin or nail that is liquid impermeable. This allows the user to apply nail polish onto an uncoated portion of nail. The coating provided by the liquid adhesive prevents any accidental application of nail polish onto the user's skin or any portions of the nail upon which the user does not wish to apply nail polish. When the user has finished applying nail polish, the quantity of rubber latex allows the user to simply peel the coating provided by the liquid adhesive off of the skin or nail. In one example embodiment of the present disclosure, the quantity of rubber latex is approximately 65.2% wt. of the liquid adhesive for nail polish. For example, the approximately 65.2% wt. of the liquid adhesive for nail polish could be anywhere between 45% wt. and 70% wt. wt. of the liquid adhesive for nail polish. In another example, the quantity of rubber latex may be greater than 50% wt. of the liquid adhesive for nail polish.

As used herein, % wt. means percentage by mass (also referred to as percentage by weight). For example, in the above embodiment with the quantity of rubber latex being approximately 65.2% wt. of the liquid adhesive for nail polish, for every 100 grams of the liquid adhesive for nail polish, approximately 65.2 grams would be rubber latex.

The quantity of water allows the liquid adhesive to maintain a liquid form prior to application to the user's nail. The liquid adhesive is primarily intended for application utilizing a brush or similar device. The quantity of liquid water maintains the liquid form of the liquid adhesive until applied. Upon exposure to air, the liquid adhesive is able to dry. In one example embodiment of the present disclosure, the quantity of water is approximately 25.6% wt. of the liquid adhesive for nail polish. For example, the approximately 25.6% wt. of the liquid adhesive for nail polish could be anywhere between 20% wt. and 45% of the liquid adhesive for nail polish. In another example, the quantity of water may be less than 50% wt. of the liquid adhesive for nail polish.

The liquid adhesive is “liquid” in that the adhesive is liquid or flowable when applied and then dries when exposed to ambient air for a period of time, for example, after application of the liquid adhesive to a fingernail or cuticle. As such, the percentage of water versus rubber latex present in the liquid adhesive may be modified to achieve desired properties for flowability during application or drying time. For example, decreasing the quantity of rubber latex and/or increasing the quantity of water present in the liquid adhesive may allow for more flowability and a longer drying time and vice versa. Additionally, the amount of rubber latex present in the liquid adhesive for nail polish may vary based on environmental conditions such as, for example, temperature and humidity. For example, cooler temperatures and/or lower humidity may decrease the flowability of the liquid adhesive during application and cause a shorter drying time and vice versa. As such, in various embodiments, the quantities of materials included in the liquid adhesive may be adjusted to achieve desired properties for flowability and/or drying time which can be affected by environmental conditions. For example, to aid in application and ease of use, the liquid adhesive preferably should flow smoothly to avoid clumps or chunkiness during application and preferably should dry shortly after, but not during application of the liquid adhesive.

In some embodiments, these environmental conditions may be based on the environmental conditions in the location where the liquid adhesive is made (e.g., where the materials forming the liquid adhesive are combined). For example, the product may be tested and modified during manufacture to achieve desired properties based on the manufacturing environment. In other embodiments, these environmental conditions may be based on the environmental conditions in the location where the liquid' adhesive is intended for use. For example, if units of the liquid adhesive are manufactured for delivery to and/or use in a warm and/or humid environment, the quantity of rubber latex may be higher than in units of the liquid adhesive manufactured for delivery to and/or use in a cooler and/or more arid environment.

The above reasons for modifying and/or adjusting the respective quantities of materials are example reasons and other reasons may be used in addition to or in lieu of the above reasons.

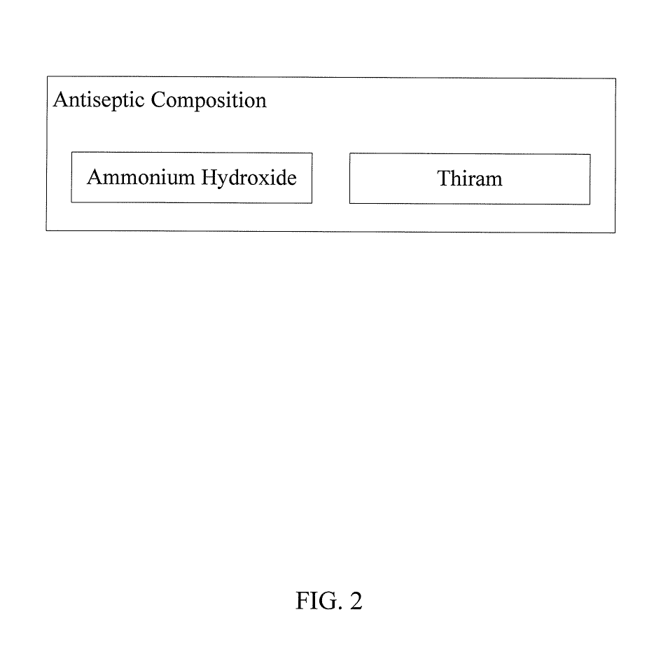

Referring again to FIG. 2 and FIG. 7, the antiseptic composition provides various antiseptic, antimicrobial, and antifungal properties to the liquid adhesive. In an illustrative embodiment, the antiseptic composition comprises a quantity of ammonium hydroxide and a quantity of thiram. The quantity of ammonium hydroxide is a solution of ammonia and water that is used in low concentrations in cosmetic applications. The quantity of ammonium hydroxide provides disinfecting properties to the portions of the user's skin or nail to which the liquid adhesive is applied. Additionally, the quantity of ammonium hydroxide further allows the liquid adhesive to maintain liquid form prior to application to the user's skin or nail. In one example embodiment, the quantity of ammonium hydroxide is approximately 0.9% wt. of the liquid adhesive for nail polish. For example, the approximately 0.9% wt. of the liquid adhesive for nail polish could be anywhere between 0.4% wt. and 1% wt. of the liquid adhesive for nail polish. Given the property of the ammonium hydroxide to allow the liquid adhesive to maintain liquid form prior to application, the quantity of ammonium hydroxide present in the liquid adhesive may be modified and/or adjusted to achieve desired properties for flowability and/or drying time as discussed above.

The quantity of thiram provides antifungal and bactericidal properties to the user's skin or nail when the liquid adhesive is applied. In one example embodiment, the quantity of thiram is approximately 0.1% wt. of the liquid adhesive for nail polish. Similar to the quantity of ammonium hydroxide, the quantity of thiram is utilized in low concentrations in cosmetic applications. In an illustrative embodiment, the quantity of thiram is present in powder, granule, or similar water-soluble form.

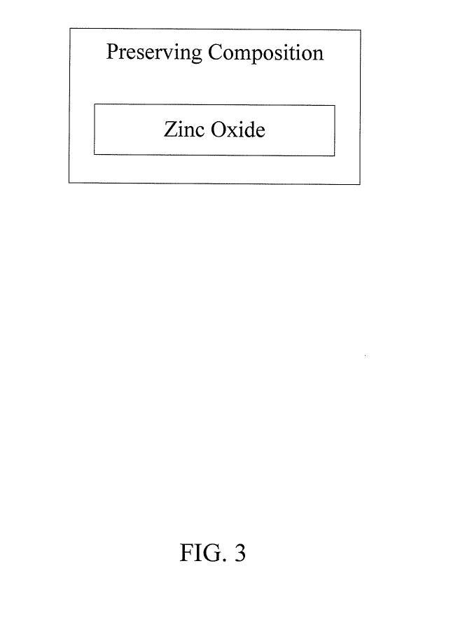

Again with reference to FIG. 3 and FIG. 7, the preserving composition preserves the liquid adhesive against decomposition, infections, and spoilage, as well as additional undesirable chemical and physical changes. In an illustrative embodiment, the preserving composition comprises a quantity of zinc oxide. The quantity of zinc oxide improves the overall microbiological quality of the liquid adhesive, greatly improving the shelf life of the liquid adhesive as well as preventing contamination of the liquid adhesive. The quantity of zinc oxide additionally provides antimicrobial and antifungal properties to the liquid adhesive. In one example embodiment, the quantity of zinc oxide is approximately 5.5% wt. of the liquid adhesive for nail polish.

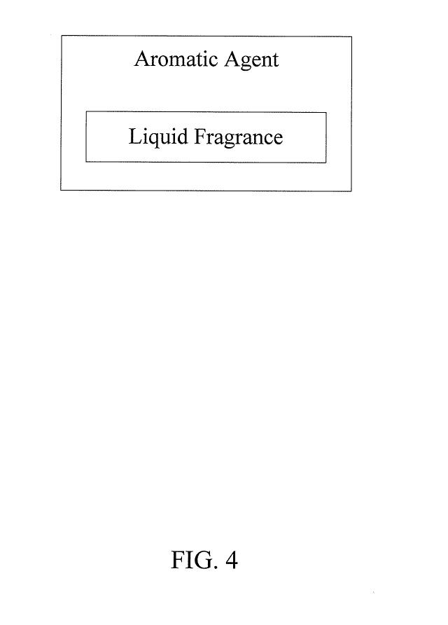

The aromatic agent serves to provide a pleasant scent to the liquid adhesive. As shown in FIG. 4, in an illustrative embodiment, the aromatic agent comprises a quantity of liquid fragrance. The embodiments of the present disclosure are not limited with respect to the specific type of scent used in the liquid adhesive nor with respect to the specific type of the quantity of liquid fragrance. For example, the quantity of liquid fragrance may be an essential oil containing aroma compounds that has been extracted from seeds, flowers, leaves, roots, stems, or bark of plants and may be an organic material. Alternatively, the quantity of liquid fragrance may be a synthetic aroma chemical. In one embodiment, the liquid fragrance may be a fragrance oil, such as coconut oil. The quantity of liquid fragrance further aids in maintaining the liquid form of the liquid adhesive. Thus, as discussed above, in some embodiments, the quantity of liquid fragrance present in the liquid adhesive may be modified and/or adjusted to achieve desired properties for flowability and/or drying time. In one example embodiment, the quantity of liquid fragrance is approximately 0.8% wt. of the liquid adhesive for nail polish as shown in FIG. 7.

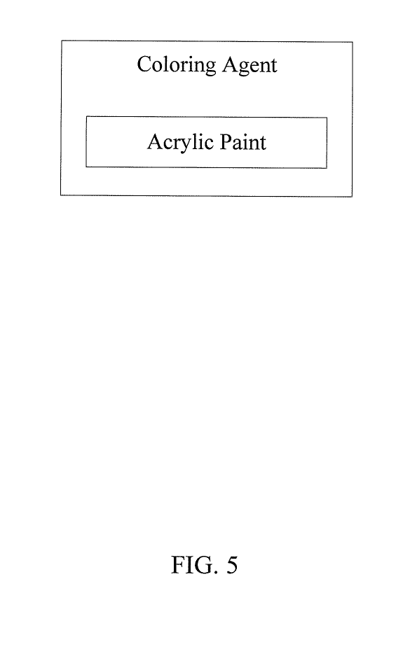

The coloring agent improves the aesthetic appearance of the liquid adhesive. As shown in FIG. 5, in an illustrative embodiment, the coloring agent comprises a quantity of acrylic paint. Similar to the quantity of liquid fragrance, the embodiments of present disclosure are not limited with respect to the specific color of the quantity of acrylic paint used in the liquid adhesive. Additionally, the quantity of acrylic paint further aids in maintaining the liquid form of the liquid adhesive. Thus, as discussed above, in some embodiments, the quantity of coloring agent present in the liquid adhesive may be modified and/or adjusted to achieve desired properties for flowability and/or drying time. In one example embodiment, the quantity of acrylic paint is approximately 1.2% wt. of the liquid adhesive for nail polish as shown in FIG. 7.

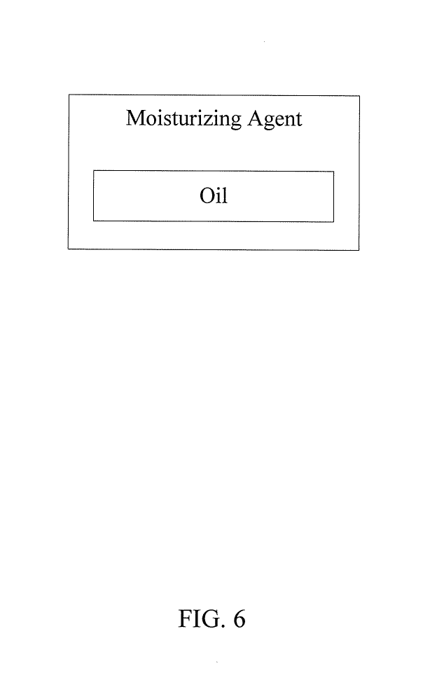

Again with reference to FIG. 6 and FIG. 7, the moisturizing agent provides moisturizing properties when applied to the user's skin. This is particularly effective when the liquid adhesive is applied to the user's cuticle as the moisturizing properties provided by the moisturizing agent are able to minimize the possibilities of hangnails and similar issues related to dry skin. In an illustrative embodiment, the moisturizing agent comprises a quantity of oil. The quantity of oil moisturizes and otherwise nourishes the user's skin when applied to the user's cuticle. In one example embodiment, the quantity of oil is approximately 0.8% wt. of the liquid adhesive for nail polish. In some embodiments, the material used for moisturizing and fragrance may be the same. For example, a fragrance oil such as, for example, coconut oil, may be used as both the liquid fragrance and the moisturizing agent. For example, the fragrance oil may be able to mask undesirable smells, provide a pleasant scent, and provide moisturization properties to prevent or reduce dehydration to the skin of the user from use of the liquid adhesive.

The example embodiments shown in FIGS. 1-7 are for the purpose of illustrating embodiments of the present disclosure and are not intended as a limitation on the number or quantity of materials that may be present or used in the liquid adhesive for nail polish of the present disclosure. For example, other materials may be present or used in addition to or instead of the materials listed in FIGS. 1-7. In another example, one or more of the materials listed in FIGS. 1-7 may be omitted in one or more embodiments. For example, in some embodiments where color and/or scent are not a concern or a low priority, one or more of the aromatic agent and the coloring agent may not be included in the liquid adhesive for nail polish. In another example, depending on antiseptic and preservation priorities, one or more of the antiseptic composition and the preserving composition may not be included in the liquid adhesive for nail polish in some embodiments. Still further, in some embodiments, the moisturizing agent may not be included in the liquid adhesive for nail polish depending, for example, on desired moisturization properties of the liquid adhesive or if the material used for the aromatic agent can provide the desired moisturization properties for the liquid adhesive.

The liquid adhesive for nail polish according to various embodiments of the present disclosure has at least three primary applications in the application of nail polish. The below examples are non-exhaustive. Other applications of the liquid adhesive may be used in addition to the examples below.

First, in various embodiments of the present disclosure, the liquid adhesive for nail polish may be applied to the cuticle surrounding the user's nail prior to the application of nail polish. The liquid adhesive is initially applied in liquid form and allowed to dry. When the liquid adhesive has been allowed to dry on the user's skin, the quantity of rubber latex forms a liquid-impermeable barrier on the user's skin. This prevents nail polish from being accidentally applied to the user's skin as the nail polish is unable to come into contact with the skin. The user is then able to apply nail polish to the surface of the nail as normal. Once dry, the liquid adhesive may simply be peeled from the skin, removing any nail polish applied on top of the coating provided by the liquid adhesive as well.

Second, in various embodiments of the present disclosure, the liquid adhesive for nail polish facilitates the removal of nail polish without the need for nail polish remover. The user is able to apply a coating of the liquid adhesive to the surface of a nail. Nail polish may then be applied as normal onto the coating of the liquid adhesive. When the user wishes to remove the nail polish, the coating of the liquid adhesive may be peeled off, removing the nail polish applied on top of the coating as well.

Third, in various embodiments of the present disclosure, the liquid adhesive for nail polish facilitates the creation of intricate nail polish designs. A first coating of nail polish is first applied to the entire surface of a nail. Once the first coating of nail polish has dried, a coating of the liquid adhesive is then applied onto the first coating of nail polish on the portions of the nail that the user does not wish to be coated by a second coating of nail polish. The coating of the liquid adhesive is allowed to dry. A second coating of nail polish is then applied over the entire surface of the nail and is allowed to dry. The coating of the liquid adhesive may then be peeled off and removed from the nail, leaving behind the first coating of nail polish and the second coating of nail polish on the user's nail in the applied design.

Although the present disclosure has been explained in relation to its preferred embodiment, it is understood that many other possible modifications and variations can be made without departing from the spirit and scope of the present disclosure as hereinafter claimed.

It may be advantageous to set forth definitions of certain words and phrases used throughout this patent document. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

Definitions for other certain words and phrases are provided throughout this patent document. Those of ordinary skill in the art should understand that in many if not most instances, such definitions apply to prior as well as future uses of such defined words and phrases.

What is claimed is:

1. A liquid adhesive for nail polish, the liquid adhesive comprising:
a quantity of rubber latex being between 45% wt. and 70% wt. of the liquid adhesive for nail polish; and
a quantity of water being between 20% wt. and 45% wt. of the liquid adhesive for nail polish.

2. The liquid adhesive for nail polish as claimed in claim 1, further comprising:
an antiseptic composition;
an aromatic agent; and
a coloring agent.

3. The liquid adhesive for nail polish as claimed in claim 2, wherein the quantity of rubber latex is greater than 50% wt. of the liquid adhesive for nail polish.

4. The liquid adhesive for nail polish as claimed in claim 2, wherein the antiseptic composition comprises a quantity of ammonium hydroxide.

5. The liquid adhesive for nail polish as claimed in claim 4, wherein the quantity of ammonium hydroxide is between 0.4% wt. and 1% wt. of the liquid adhesive for nail polish.

6. The liquid adhesive for nail polish as claimed in claim 2, further comprising a moisturizing agent.

7. The liquid adhesive for nail polish as claimed in claim 6, wherein the moisturizing agent comprises fragrance oil.

8. The liquid adhesive for nail polish as claimed in claim 7, wherein the fragrance oil is both the aromatic agent and the moisturizing agent.

9. The liquid adhesive for nail polish as claimed in claim 8, wherein the fragrance oil comprises coconut oil.

10. The liquid adhesive for nail polish as claimed in claim 2, further comprising a preserving composition.

11. The liquid adhesive for nail polish as claimed in claim 10, wherein the preserving composition comprises zinc oxide.

12. The liquid adhesive for nail polish as claimed in claim 2, wherein the antiseptic composition comprises a quantity of thiram

13. The liquid adhesive for nail polish as claimed in claim 2, wherein the coloring agent comprises a quantity of acrylic paint.

14. A liquid adhesive for nail polish, the liquid adhesive comprising:
a quantity of rubber latex being between 45% wt. and 70% wt. of the liquid adhesive for nail polish; and
a quantity of water being between 20% wt. and 45% wt. of the liquid adhesive for nail polish,
wherein the quantity of rubber latex and the quantity of water present in the liquid adhesive for nail polish is selected based on environmental conditions.

15. The liquid adhesive for nail polish as claimed in claim 14, wherein the quantity of rubber latex and the quantity of water present in the liquid adhesive for nail polish is selected based on environmental conditions in a location where the liquid adhesive for nail polish is made.

16. The liquid adhesive for nail polish as claimed in claim 14, wherein the quantity of rubber latex and the quantity of water present in the liquid adhesive for nail polish is selected based on environmental conditions in a location where the liquid adhesive for nail polish is intended for use.

17. The liquid adhesive for nail polish as claimed in claim 14, wherein the environmental conditions comprise temperature and humidity.

18. The liquid adhesive for nail polish as claimed in claim 14, further comprising a quantity of antiseptic composition, wherein the quantity of antiseptic composition in the liquid adhesive for nail polish is selected based on the environmental conditions.

19. A liquid adhesive for nail polish, the liquid adhesive comprising:
a quantity of rubber latex that is greater than 50% wt. of the liquid adhesive for nail polish;
a quantity of water that is less than 50% wt. of the liquid adhesive for nail polish.

20. The liquid adhesive for nail polish as claimed in claim 19, further comprising:
an antiseptic composition;
a preserving composition;
an aromatic agent;
a coloring agent; and
a moisturizing agent.
